(19) United States
(12) Patent Application Publication
(10) Pub. No.: US 2026/0166567 A1
(43) Pub. Date: Jun. 18, 2026

(54) SYSTEM AND METHOD FOR FLUIDICS CARTRIDGE COMPRISING SEMICONDUCTOR ARRAY FOR BIOMARKER DETECTION

(71) Applicant: Bryan Joseph Rice, San Diego, CA (US)

(72) Inventor: Bryan Joseph Rice, San Diego, CA (US)

(21) Appl. No.: 19/383,557

(22) Filed: Nov. 7, 2025

Related U.S. Application Data
(60) Provisional application No. 63/718,530, filed on Nov. 8, 2024.

(51) Int. Cl. B03C 5/02; B01L 3/00; B03C 5/00; G01N 27/447

(52) U.S. Cl. CPC B03C 5/026; B01L 3/502715; B03C 5/005; G01N 27/44791; B01L 2200/027; B01L 2200/0689; B01L 2200/16; B01L 2300/044; B01L 2300/0645; B01L 2300/18; B01L 2400/0487

(57) ABSTRACT

The present invention is directed to a system and method for a research instrument device. A method of purifying extracellular vesicles (“EVs”) is disclosed wherein a biological sample including EVs is obtained and applied to a fluidic cell in an array of fluidic cells of a multistage dielectrophoretic (“DEP”) filter system. The DEP filter system comprises a semiconductor array comprising a plurality of electrodes. The DEP filter system is disposed within a fluidics system; wherein the fluidics system is configured to move at least one fluid across the semiconductor array of the DEP filter system.

### CROSS REFERENCE TO RELATED APPLICATIONS

This application claims the benefit of U.S. Provisional Patent Application No. 63/718,530, filed Nov. 8, 2024, the contents of which are incorporated herein by reference.

### BACKGROUND OF THE INVENTION

The present invention is directed to a system and method for a research instrument device. There is a growing demand in the field of diagnostics for early disease detection that is fast, accurate, and sensitive to low quantities of biomarkers. Early detection of diseases including cancer and neurodegenerative diseases can significantly increase the likelihood of survivability and successful treatment of the disease.

Biomarkers are measurable characteristics produced by the body that can be used to indicate a normal or abnormal stasis. Extracellular vesicles (“EVs”) are nano-sized, membrane-bound particles released by cells and cellular components throughout the body and play a key role in intercellular communication via their biomarker components. EVs are present in biological fluids including blood, urine, cerebrospinal fluid, etc.

Dielectrophoresis (“DEP”) can be employed to capture EVs of interest for use in diagnostic procedures. DEP works by exerting a force on a dielectric particle when it is subjected to a non-uniform electric field. DEP does not require that the particle be a charged particle, as all particles exhibit dielectrophoretic activity in the presence of electric fields. The present invention takes advantage of the particle-capturing abilities of DEP in the collection of EVs.

### SUMMARY OF THE INVENTION

The present invention pertains to a system and method for early disease detection. In particular embodiments of the present invention, a method of purifying extracellular vesicles (“EVs”) is disclosed wherein a biological sample including EVs is obtained and applied to a fluidic cell in an array of fluidic cells of a multistage dielectrophoretic (“DEP”) filter system.

The present invention uses dielectrophoresis (“DEP”) to preferentially collect specific particles from a biological sample. The type of particles collected are driven by the parameters used to define the electric field used in DEP. The device passes the samples and various reagents across a patterned silicon die capable of creating the electric fields used within the DEP process. The die is mounted in a processing chamber that controls the fluid volume exposed to the DEP and performs micro-mixing.

An electric field capable of performing DEP is created using a signal generation IC. The resulting waveform is passed through a gain and shaping analog chain before being amplified into a high-power signal applied to the chip. Inline metrology is performed at generation load time to ensure the correct frequency components and voltage amplitude are present as required by the DEP parameters.

Fluidic control is handled by a robotic pipette handler capable of selecting, delivering, and moving fractional microliters of the sample/reagents stored on a disposable process cartridge. Various valves and pumps are controlled from FW to allow precise volume and flowrate desired to be present in the process chamber while the DEP is active. Waste fluids are removed and disposed of in a safe and automated manner. The hardware component of the instrument is designed to allow reconfiguration of frequency and voltage to each of the various fluid ICs present in the system.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The various embodiments are illustrated by way of example, and not by way of limitation, in the figures of the accompanying drawings. Having thus described the invention in general terms, reference will now be made to the accompanying drawings, which are not necessarily drawn to scale, and wherein:

FIG. 1 shows the architecture of the present invention.

FIGS. 2A-H show the general design of the cartridge and flow cell.

FIG. 3 shows an example assay workflow within the flow cell

FIGS. 4A-E show the fluid workflow steps.

FIGS. 5A-H show the instrument workflow step 1.

FIGS. 6A-C show the instrument workflow steps 2-4.

FIG. 7 shows the materials of the present invention.

FIGS. 8A-G show the workflow and method of the present invention.

FIG. 9 shows the biomolecule contaminant removeable (e.g. lipoprotein immunodepletion).

FIG. 10 shows a bulk biomarker detection method chart.

FIGS. 11A-D show exemplary views of the semiconductor array.

FIGS. 12A-I show variations of the semiconductor array.

### DETAILED DESCRIPTION OF THE PREFERRED EMBODIMENT

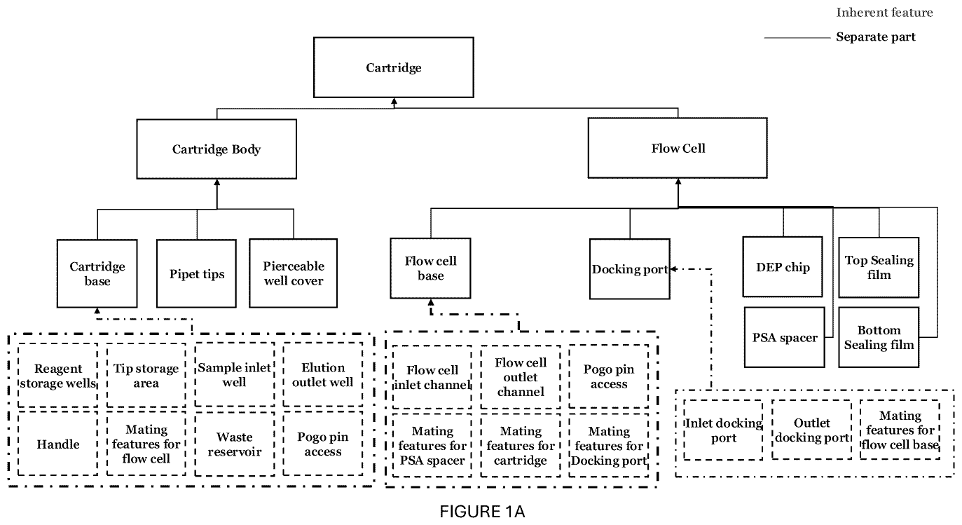

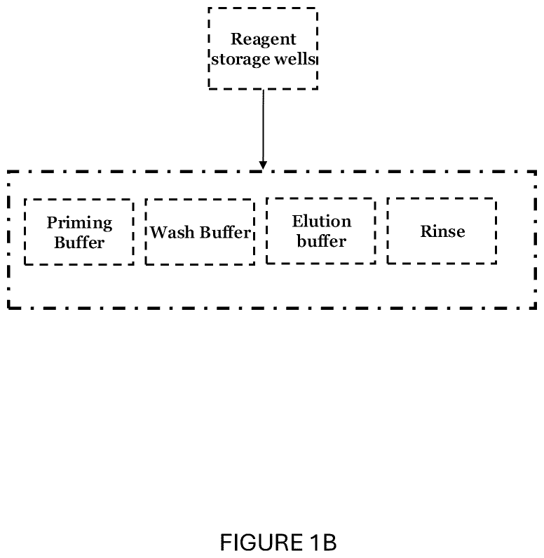

FIG. 1A shows the architecture of the present invention. In accordance with the preferred embodiment of the present invention, the cartridge comprises the cartridge body and the flow cell. The cartridge body comprises a cartridge base, a plurality of pipette tips, and a pierceable well cover. The cartridge base comprises a plurality of separate parts, including but not limited to reagent storage wells, tip storage area, sample inlet well, elution outlet well, a handle, mating features for flow cell, a waste reservoir, and pogo pin access. The flow cell comprises the flow cell base, which comprises a plurality of separate parts including a flow cell inlet channel, a flow cell outlet channel, a pogo pin access, mating features for PSA spacer, mating features for cartridge, and mating features for docking port. The flow cell further comprises a docking port, which comprises a plurality of optional features including inlet docking ports, outlet docking ports, and mating features for flow cell base. The flow cell further comprises the dielectrophoresis (“DEP”) chip, PSA spacer, top sealing film, and bottom sealing film.

FIG. 1B shows the architecture of the separate components of the reagent storage wells. In accordance with the preferred embodiment of the present invention, the reagent storage wells further comprise a plurality of separate components including but not limited to priming buffer, wash buffer, elution buffer, and rinse.

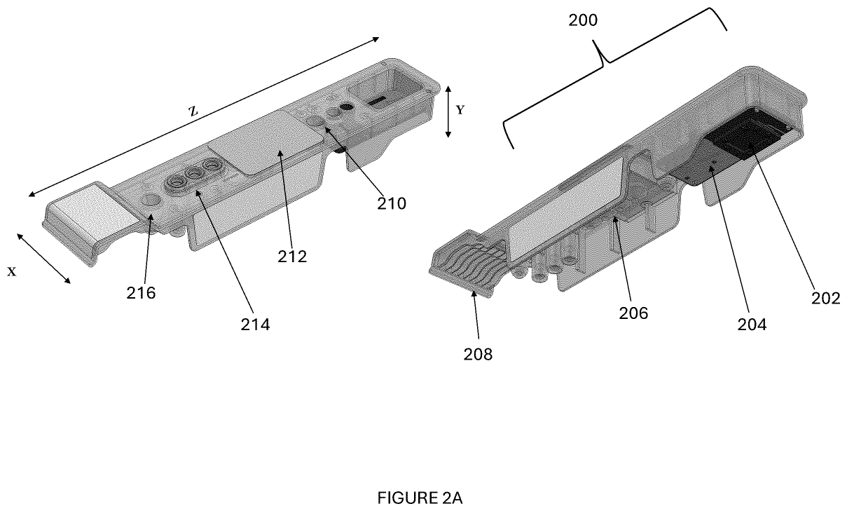

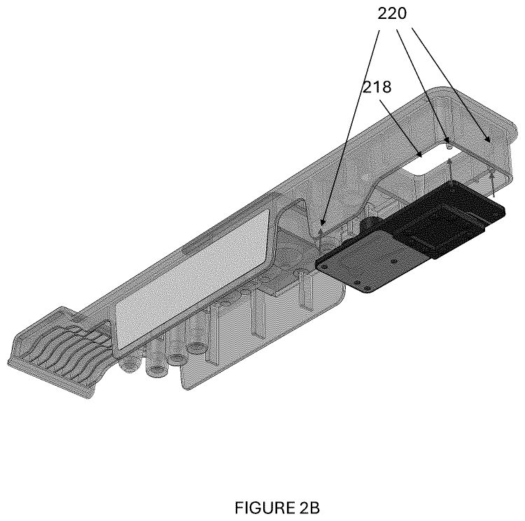

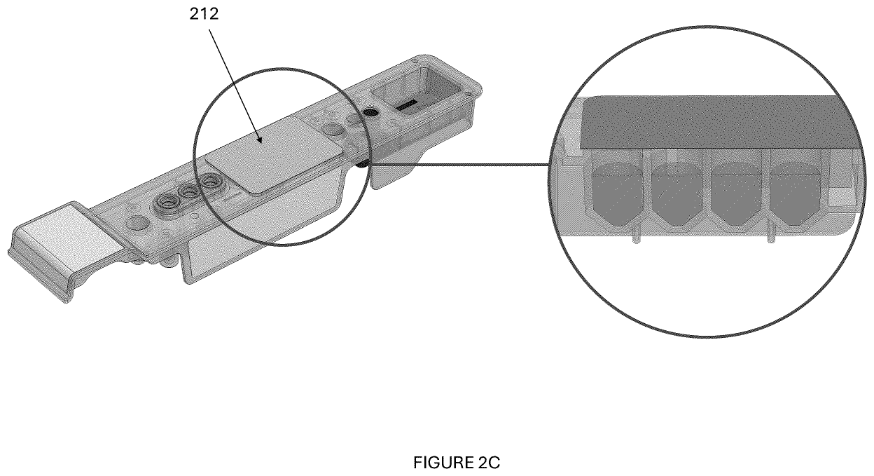

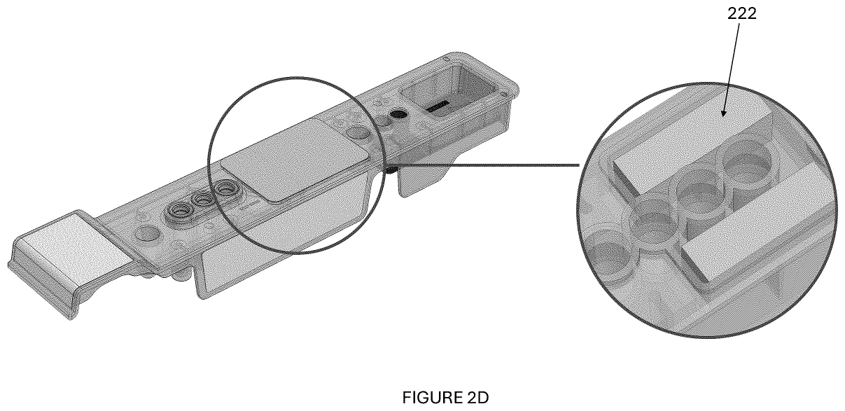

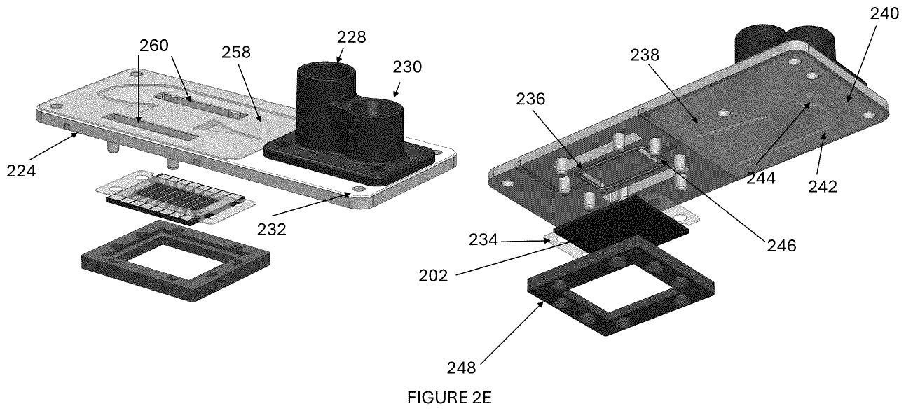

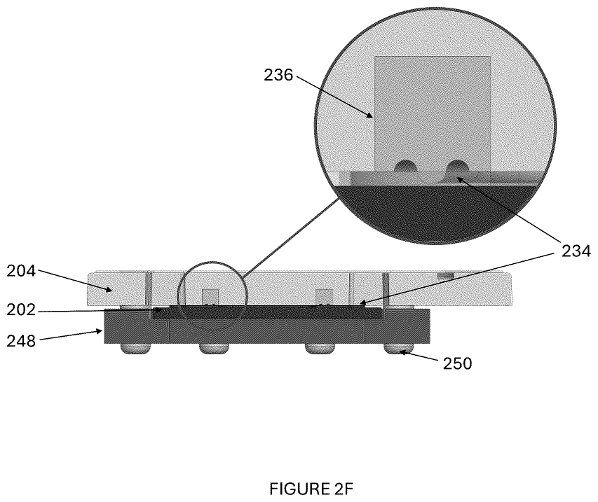

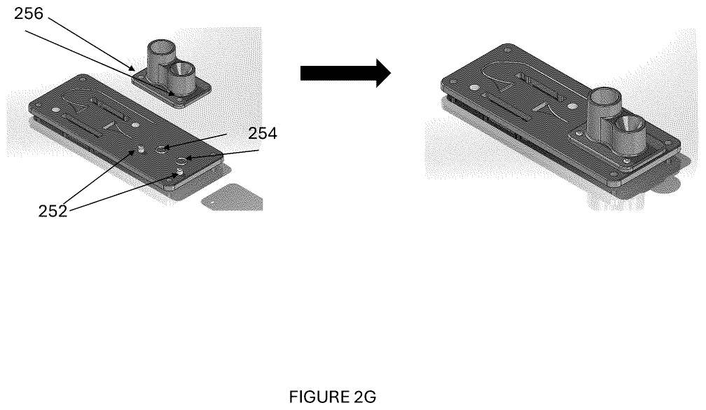

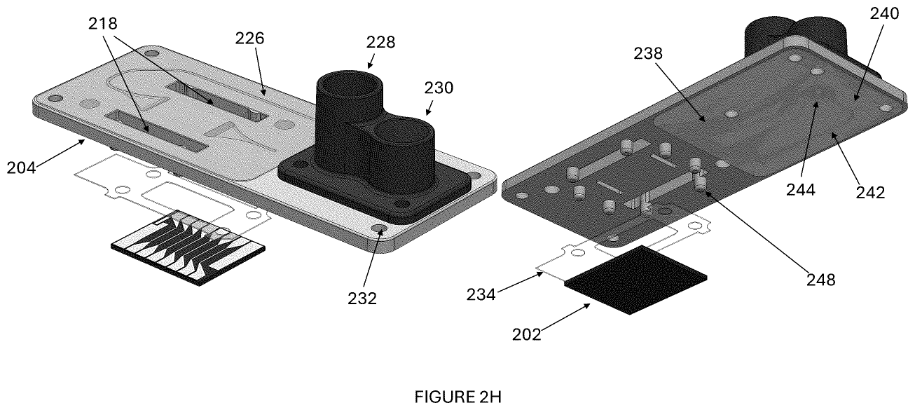

FIGS. 2A-D show the general design of the cartridge. In accordance with the preferred embodiment of the present invention, the cartridge 200 has a length of Z, a width of X, and a height of Y. The cartridge may comprise an elution outlet well 216, a plurality of pipette tips and tip storage areas 214, a pierceable well cover 212, a sample inlet well 210, a handle 208, a plurality of reagent storage wells 206, a flow cell 204 and a DEP chip 202. In order to allow for interface with the flow cell 204, the cartridge 200 further comprises mating features 220 for the flow cell 204 and pogo pin access 218. Under the pierceable well cover 212 are the plurality of reagent storage wells 206 as shown in FIG. 2C. Adjacent to the reagent storage wells 206 is the waste reservoir 222 for liquid waste. Optionally, the waste reservoir 222 may comprise a sponge to absorb waste liquid to prevent spillage.

FIGS. 2E-H show the general design of the flow cell. In accordance with the preferred embodiment of the present invention, the flow cell may comprise a flow cell base 224, pogo pin access 260, top sealing film 258, inlet docking port 228, outlet docking port 230, and mating features for cartridge 232. In some embodiments, the flow cell further comprises overmold 236 which provides a seal, flow cell inlet channel 238, bottom sealing film 240, flow cell outlet channel 242, pinch valve 244, mating features for PSA 246, an optional chip retention frame 248, DEP chip 202, and PSA spacer 234. The flow cell further comprises heat stakes 250 which provide temperature control. FIG. 2G shows additional mating features 256, 254, and 252 for the dock port to connect to the flow cell. Mating features 254 may be ribbed for sonic welding. Mating features 252 may be designed for specific alignment of the attachment.

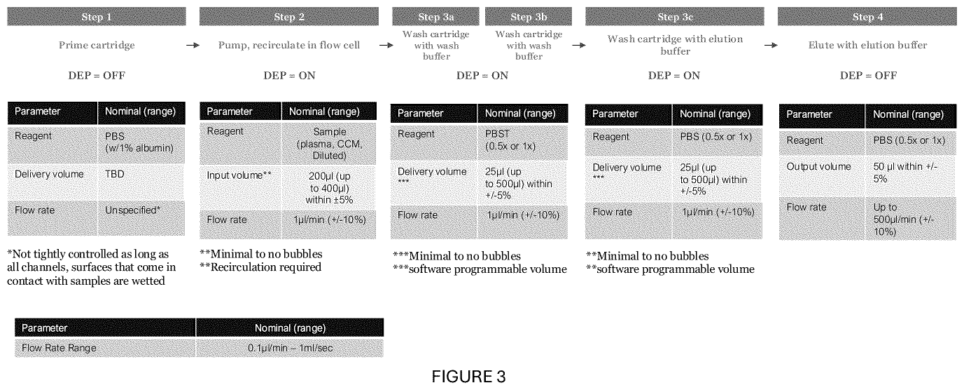

FIG. 3 shows an example assay workflow within the flow cell. In accordance with the preferred embodiment of the present invention, the flow cell process may contain steps 1-4. Step 1 may comprise priming the cartridge. During step 1, DEP is off. The parameters of step 1 may include reagent, delivery volume, and flow rate. Step 2 may comprise pumping and recirculating in the flow cell. During step 2, DEP is on. The parameters of step 2 may comprise reagent, which may be a biological sample including but not limited to blood, plasma, urine, cerebrospinal fluid, or other bodily fluid, input volume, and flow rate. Steps 3a-b may comprise washing the cartridge with wash buffer. During steps 3a-b, DEP is on. The parameters of steps 3a-b may comprise reagent, delivery volume, and flow rate. Step 3c may comprise washing the cartridge with elution buffer. During step 3c, DEP is on. The parameters of step 3c may comprise reagent, delivery volume, and flow rate. Step 4 may comprise eluting the cartridge with elution buffer. During step 4, DEP is off. The parameters of step 4 may include reagent, output volume, and flow rate.

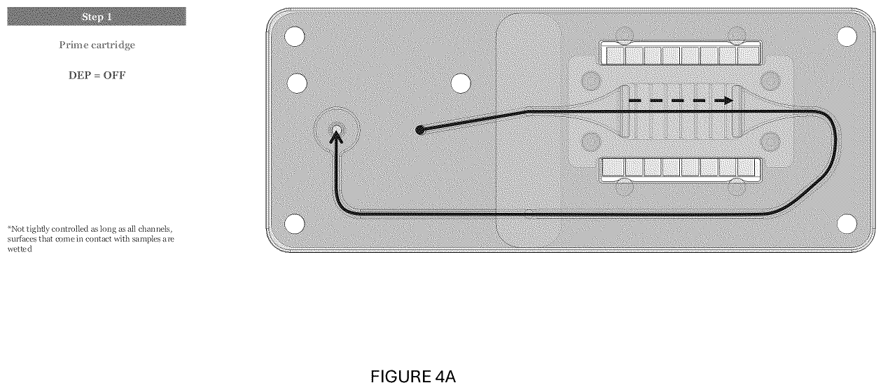

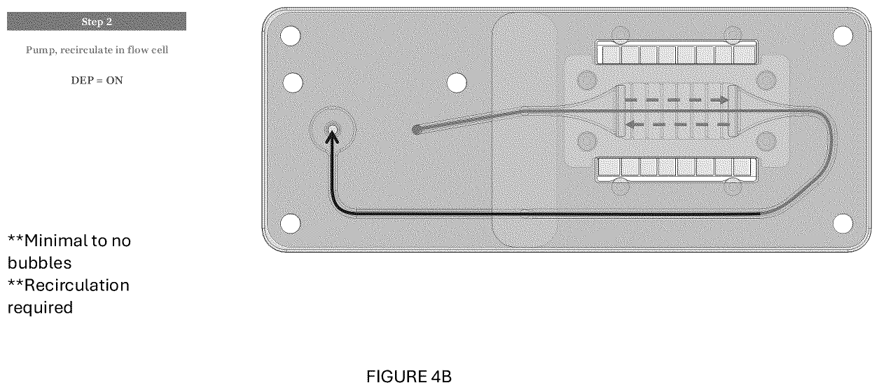

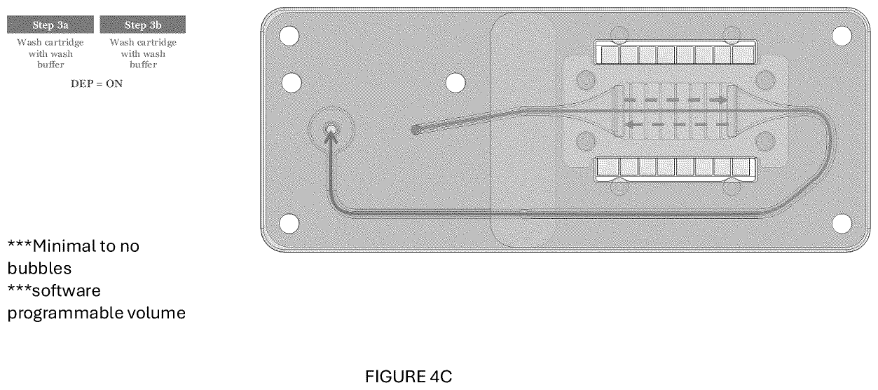

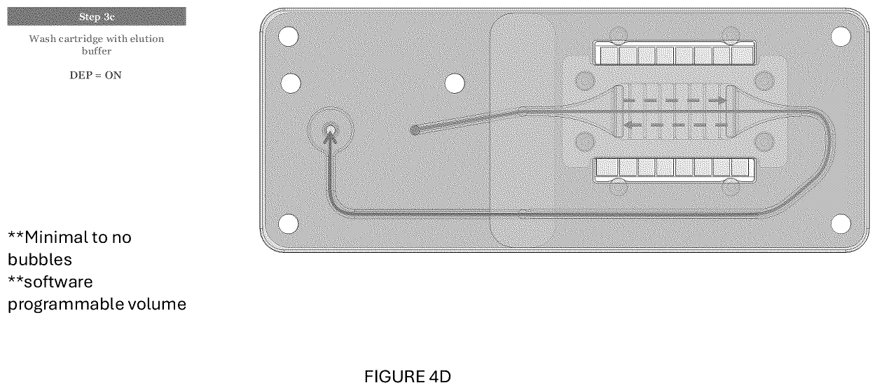

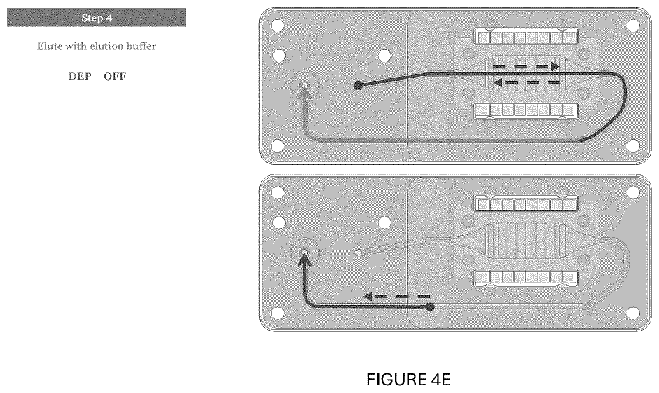

FIGS. 4A-E shows the fluid workflow steps. In accordance the preferred embodiment, in step 1, the cartridge is primed. The solid arrow of FIG. 4A shows the flow of prime buffer through the flow cell. The dashed arrow of FIG. 4A shows the movement specifically across the DEP chip. FIG. 4B shows step 2 of the process. During step 2, a pipette is used to pump the fluid throughout the flow cell. The dashed arrows of FIG. 4B show the back-and-forth movement of liquid as it is pumped across the DEP chip. This ensures that the fluid, which in the case of step 2, is a biological sample, is thoroughly moved across the DEP chip in order to ensure capture of EVs or other biomarkers of interest. FIG. 4C shows steps 3a-b. Similar to step 2, fluid is moved across the DEP chip in a back-and-forth motion via pulling and releasing of a pipette. FIG. 4D shows similar movement to FIGS. 4B and 4C of the elution buffer across the DEP chip in accordance with step 3C. Step 4 is shown in FIG. 4E. As shown, fluid is removed from the cartridge after the elution buffer is eluted.

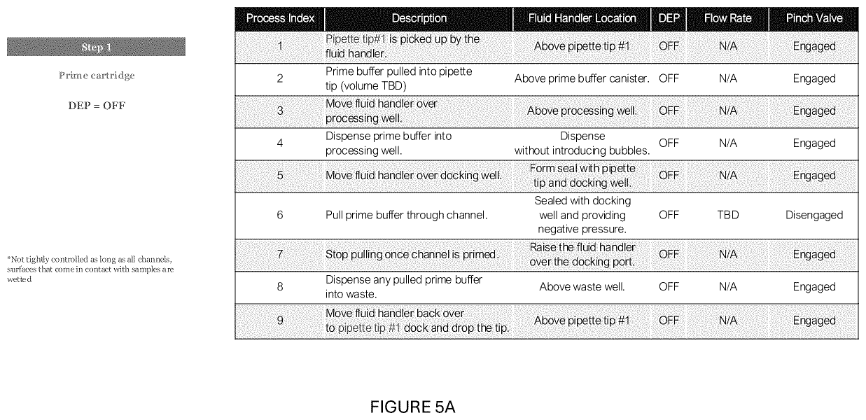

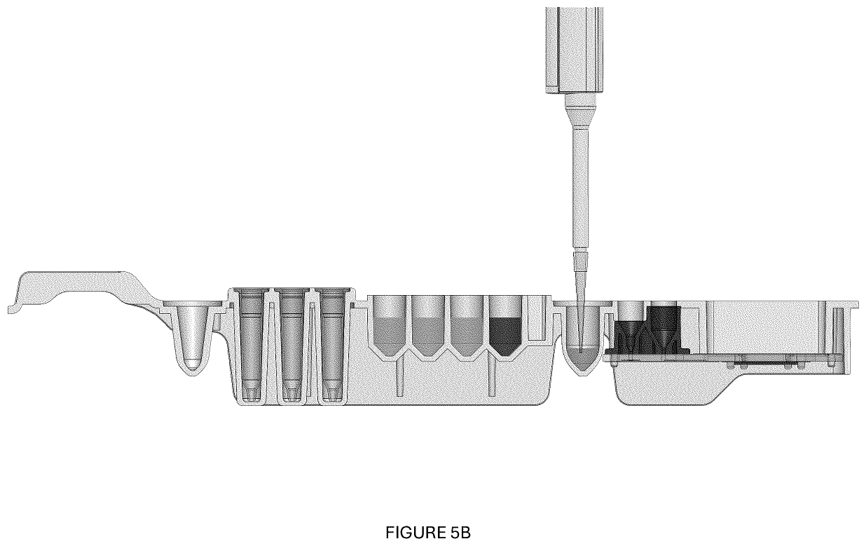

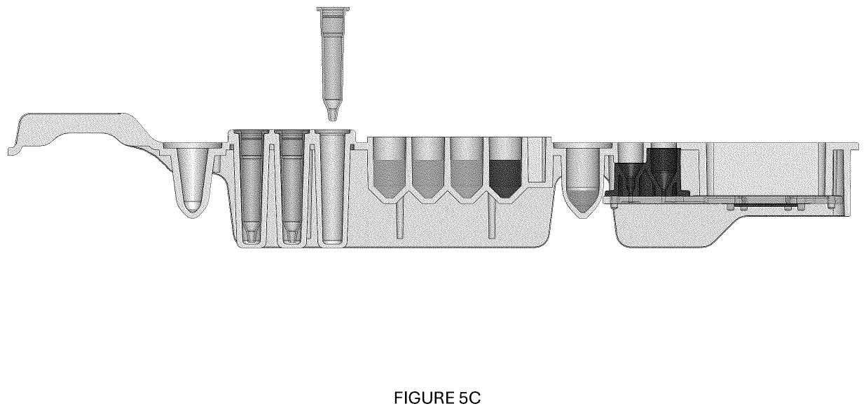

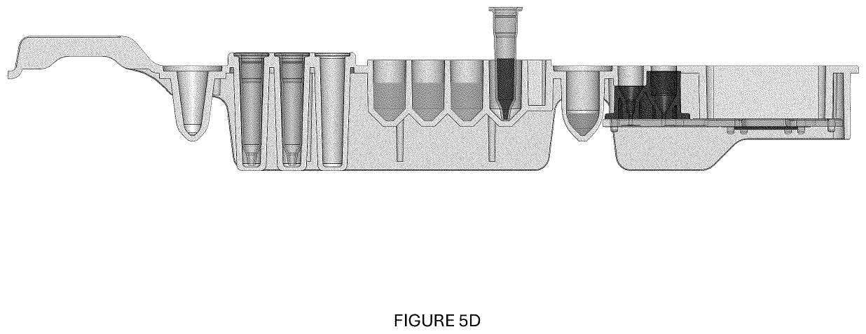

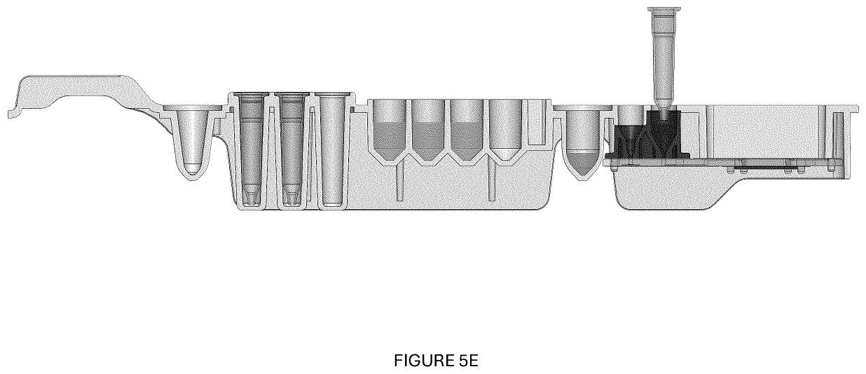

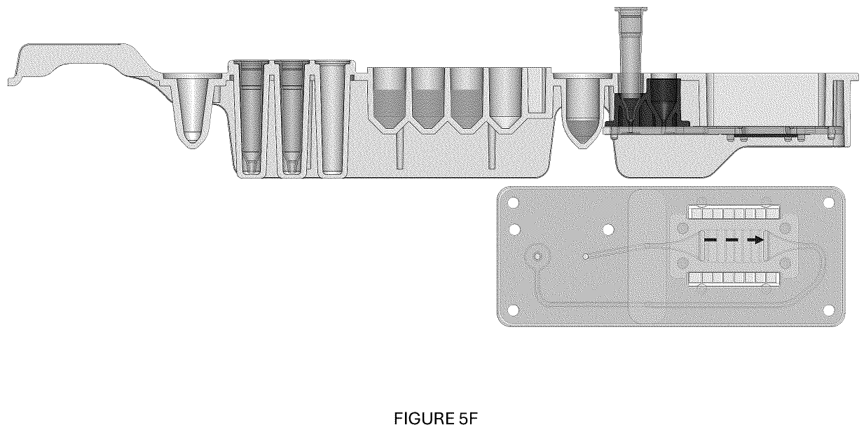

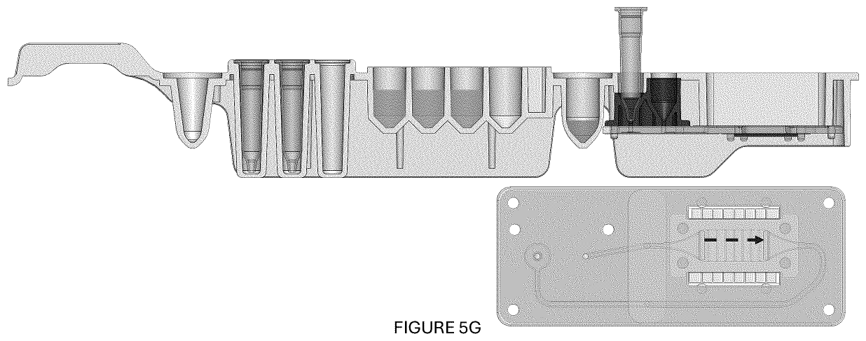

FIGS. 5A-H shows the instrument workflow step 1. In accordance with the preferred embodiment of the present invention, the process begins when a user pipettes in the sample fluid into the appropriate sample well. Then pipette tip #1 is picked up by a fluid handler. The fluid handler may be an automated system designed to automate the use of the pipette. With pipette tip #1, prime buffer is pulled into the pipette tip from the prime buffer well. With the prime buffer in pipette tip #1, the fluid handler moves the pipette to the processing well and dispenses the prime buffer into the processing well. The fluid handler then moves to the docking well, where the pipette will pull the prime buffer through the channel. Once the prime buffer has been pulled through the channel, any prime buffer that has been pulled into pipette tip #1 will be dispensed into the waste reservoir. Once pipette tip #1 has been emptied of all waste liquid, the fluid handler dispenses pipette tip #1 back in the location it was picked up from and moves on to repeat the process with the next pipette tip and the sample fluid.

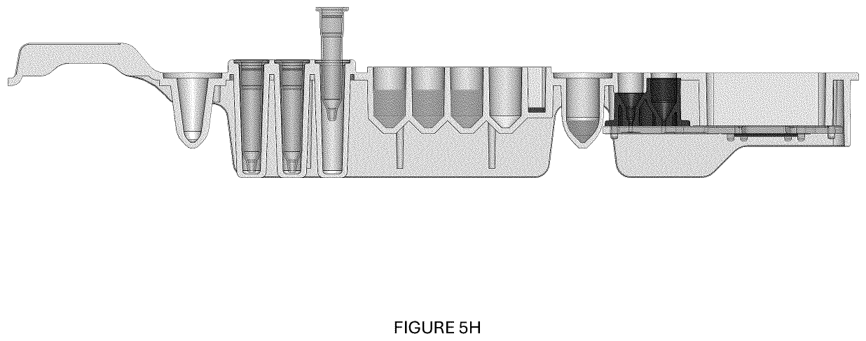

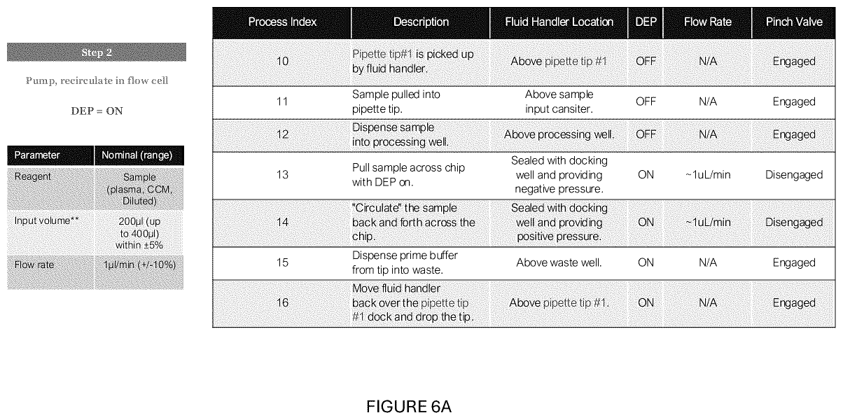

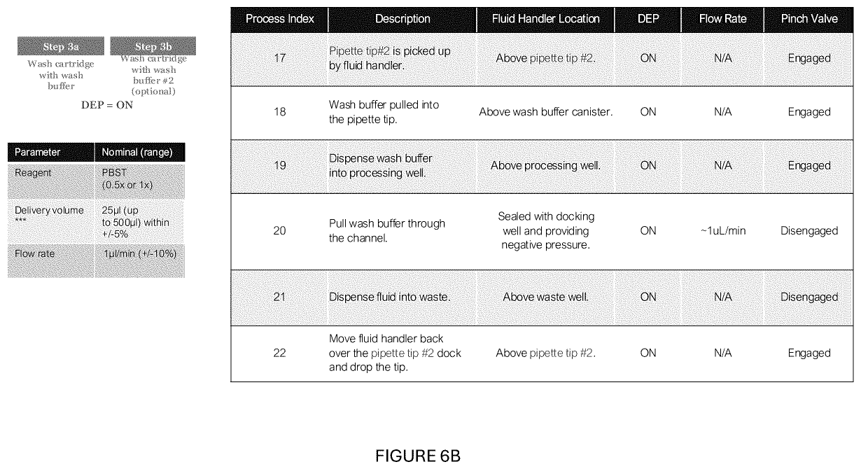

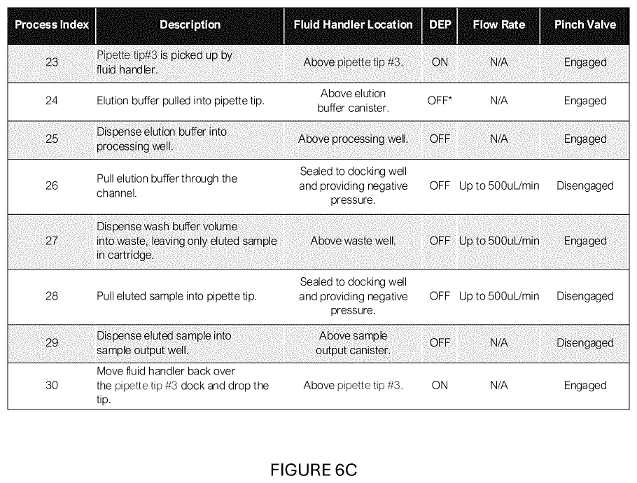

FIGS. 6A-C show the instrument workflow steps 2-4. In accordance with the preferred embodiment of the present invention, step 2 is performed with pipette tip #1. Similar protocol as to that followed in step 1 is repeated, with the exception that the liquid pulled by the pipette is the sample liquid. Additionally, in step 2, the pipette is used to pump the sample through the fluidics channels to ensure the sample is thoroughly distributed across the DEP chip. Similar protocol is followed in steps 3a-c and step 4 as shown in FIGS. 6B-C.

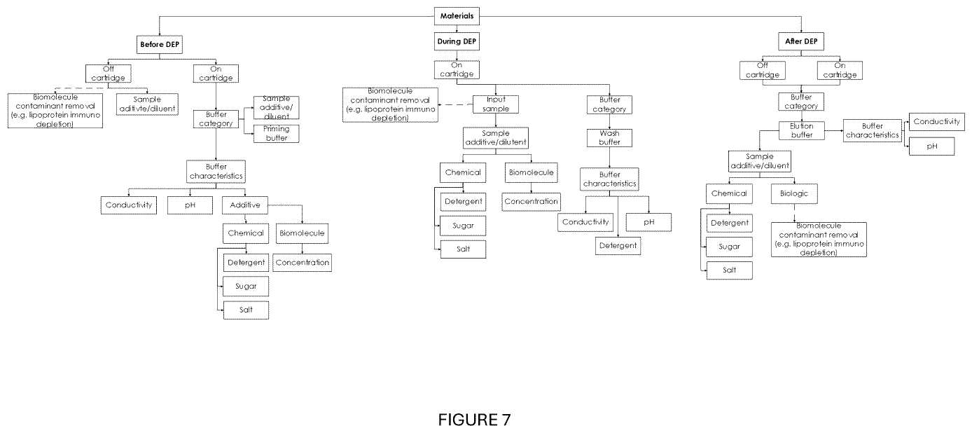

FIG. 7 shows the materials of the present invention. In accordance with the preferred embodiment of the present invention, the materials before DEP, off cartridge, comprise the sample additive or dilutant, and separately, the biomolecule contaminant removal (for example, and not by way of limitation, lipoprotein immunodepletion). On cartridge, the buffer category comprises the sample additive or dilutant, wherein buffer characteristics including but not limited to conductivity, pH, and additive characteristics including chemical, which further comprises detergent, sugar, and salt characteristics or biomolecule, which further comprises concentration characteristics.

During DEP, on cartridge, the materials comprise the input sample and the buffer category. The input sample may comprise sample additive or dilutant, which may comprise chemical or biomolecule characteristics as previously disclosed. The buffer may be a wash buffer, which may comprise the characteristics of conductivity, detergent, and pH as previously disclosed. After DEP, off or on cartridge, the materials comprise of the buffer category as previously disclosed.

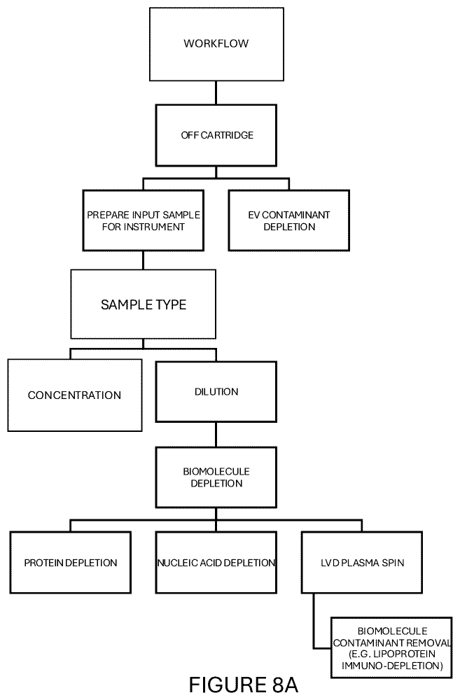

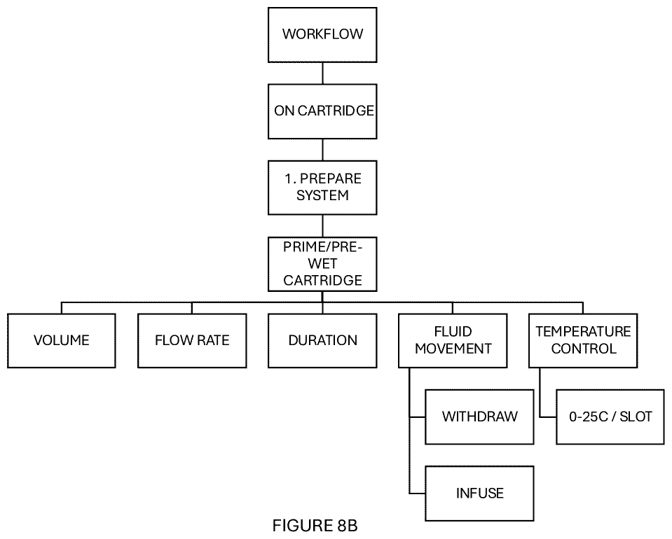

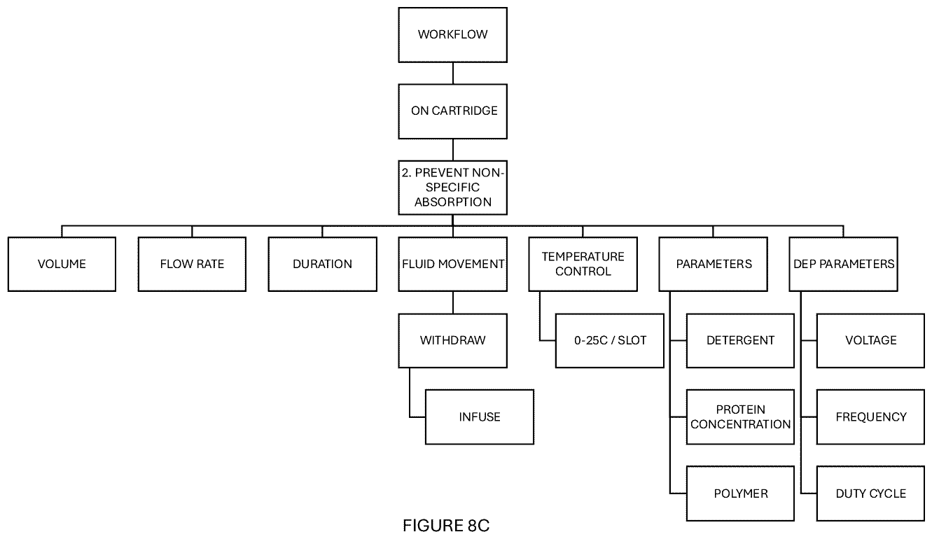

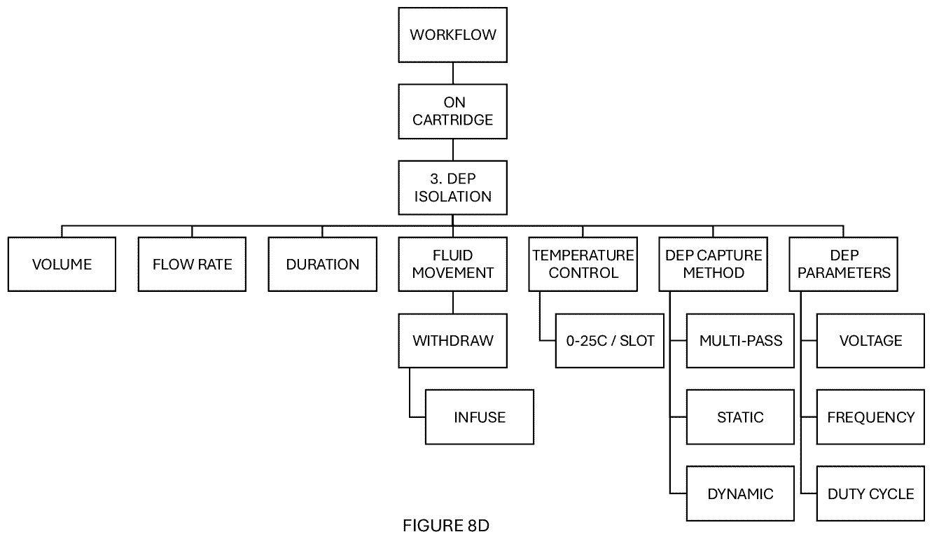

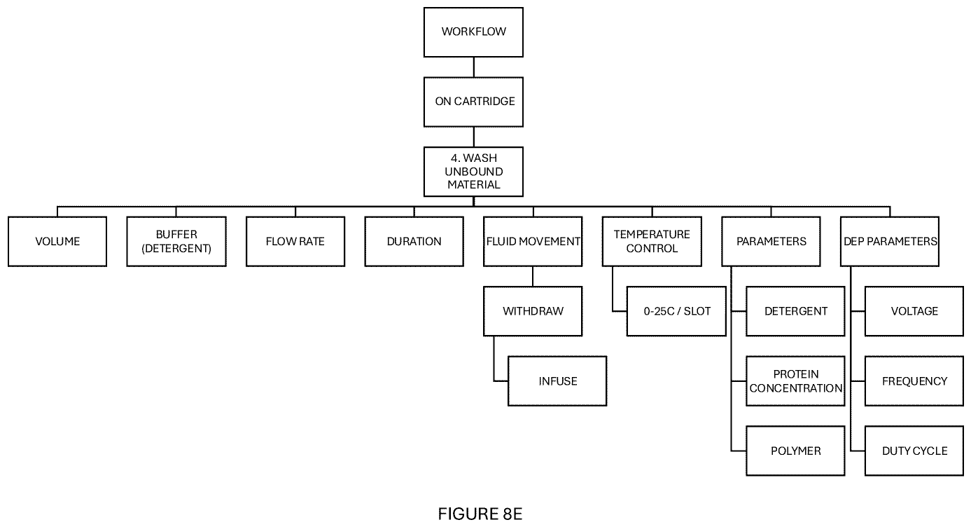

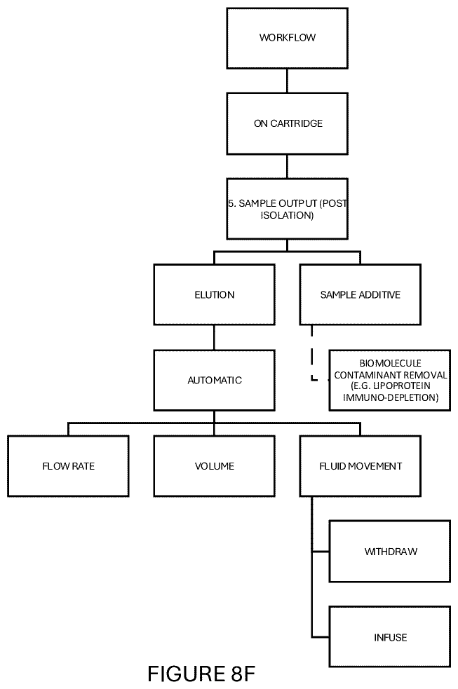

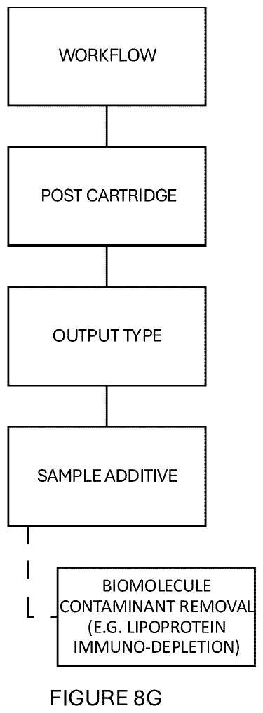

FIGS. 8A-G show the workflow and method of the present invention. In accordance with the preferred embodiment of the present invention, the off-cartridge workflow begins with preparing the input sample for the instrument and EV contaminant depletion. Following preparation of the sample, which may be of a variety of sample types including but not limited to EDTA plasma, blood, concentrated conditioned media, serum, saliva, urine, cerebrospinal fluid, or other biological sample, the sample is diluted and undergoes biomolecule depletion, which may comprise protein depletion, nucleic acid depletion, or LVD plasma spin. The on-cartridge workflow begins with preparing the system and priming the cartridge. Parameters for priming the cartridge include but are not limited to volume, flow rate, duration, fluid movement (withdraw and infuse), and temperature control. Temperature may be kept between 0-25 degrees Celsius per slot. The second step in the on-cartridge workflow is the prevention of non-specific absorption. The third on cartridge step comprises DEP isolation. Parameters for DEP isolation include volume, flow rate, duration, fluid movement, temperature control, DEP capture method (which may include but is not limited to multi-pass, static, or dynamic) and DEP parameters which may include but are not limited to voltage, frequency, and duty cycle. Following DEP, unbound material is washed from the flow cell and any remaining liquid is removed.

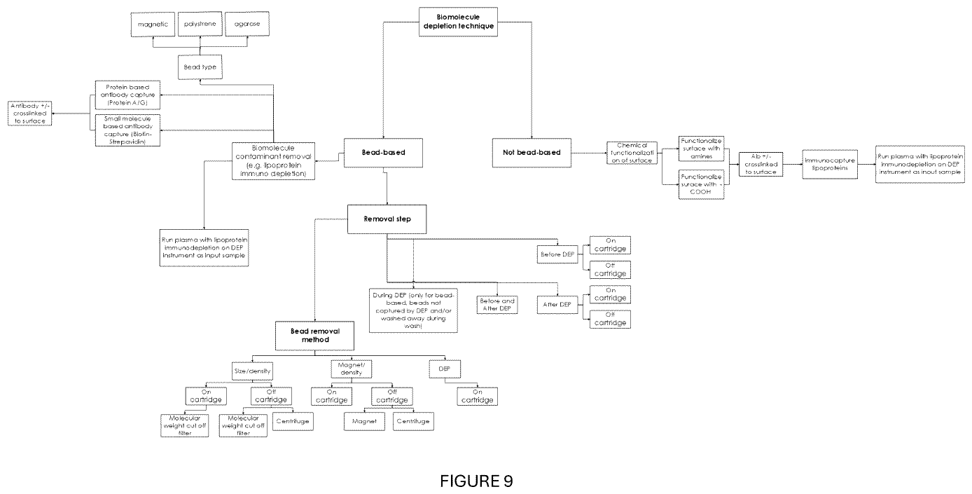

FIG. 9 shows the biomolecule depletion technique. In accordance with the preferred embodiment of the present invention, biomolecule depletion technique may be bead-based or not bead-based. Bead-based depletion may comprise biomolecule contaminant removal (for example, and not by way of limitation, lipoprotein immunodepletion) which may involve bead types including magnetic, polystyrene, or agarose. Biomolecule contaminant removal may comprise protein-based antibody capture or small molecule-based antibody capture. Biomolecule contaminant removal may comprise running plasma with lipoprotein immunodepletion on the DEP instrument as input sample. The bead-based removal step may comprise a bead removal method, which may further comprise removal based on size density, magnetic density, or DEP. Size density bead removal may occur on or off cartridge via molecular weight cut off filter, or off cartridge via centrifuge. Magnetic density removal may occur on cartridge or off cartridge via magnet or centrifuge. DEP bead removal may occur on cartridge.

Not bead-based biomolecule depletion techniques may include chemical functionalization on surface, which may comprise functionalized surface with amines or functionalized surface with COOH. Either functionalization with amines or COOH may comprise ab positive or negative cross-linkage to surface, which may comprise immunocapture of lipoproteins, which may further comprise running plasma with lipoprotein immunodepletion on DEP instrument as input sample.

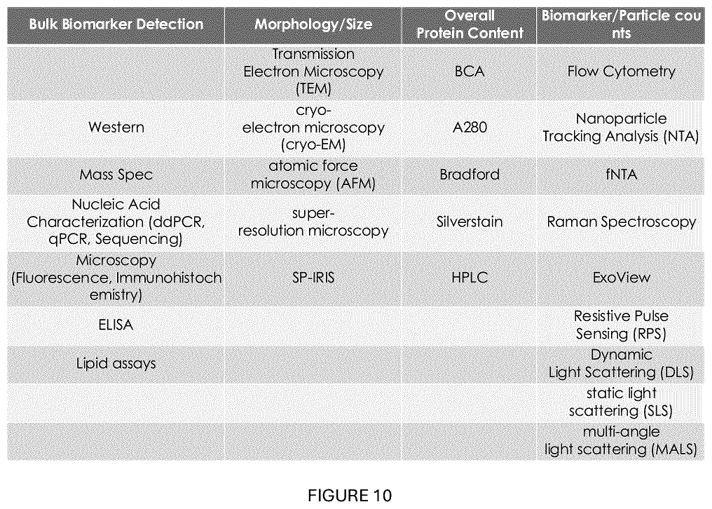

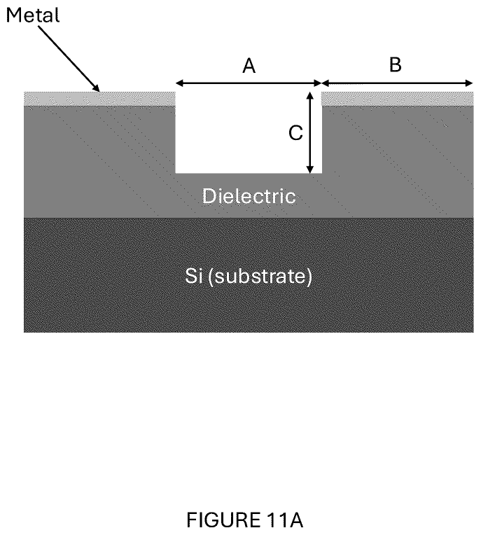

FIG. 10 shows a bulk biomarker detection method chart. In accordance with the preferred embodiment of the present invention, a plurality of bulk biomarker detection methods are disclosed, including western blot, pass spectroscopy, nucleic acid characterization, microscopy, ELISA, and lipid assays.

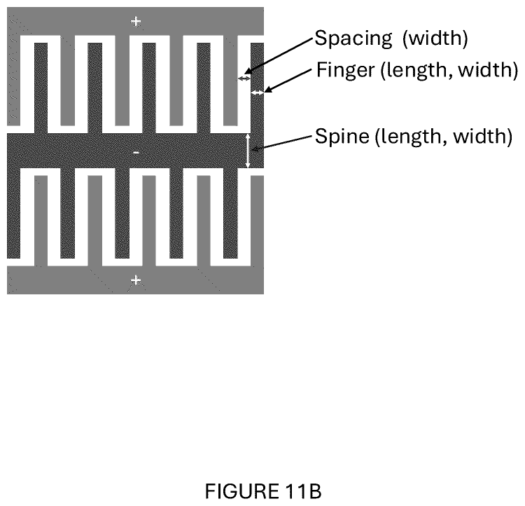

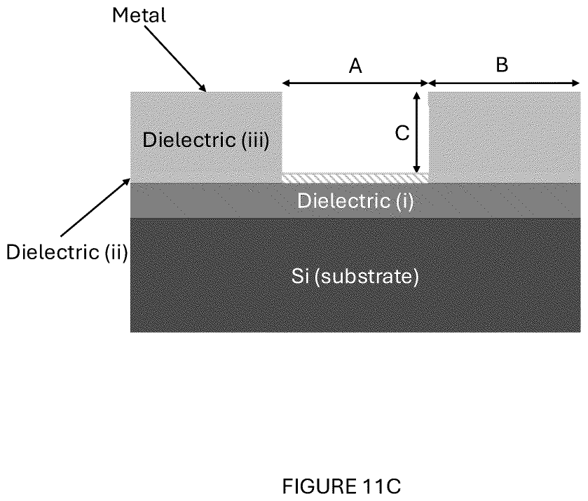

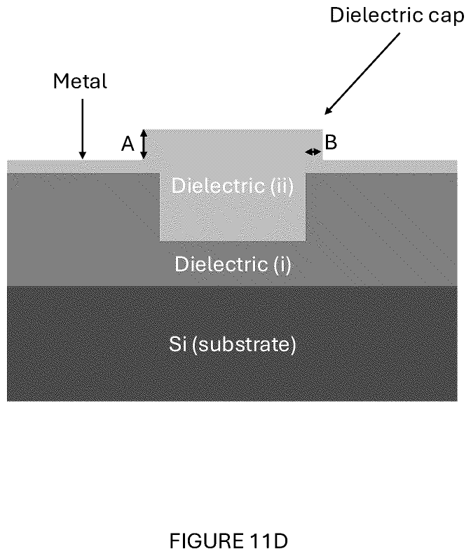

FIGS. 11A-D show exemplary views of the semiconductor array. FIG. 11A shows a cross section of a standard DEP stack. In accordance with the preferred embodiment of the present invention, a semiconductor array comprising a plurality of electrodes is disposed within a fluidics system, wherein said fluidics system is configured to move at least one fluid across the semiconductor array. The semiconductor array containing the plurality of electrodes may comprise of three layers, a first layer of metal, a second layer of a dielectric material, and a third layer of substrate. The dielectric layer may be designed in a way such that raised portions of the dielectric are interdigitated forming grooves if depth C and width A, interspaced by raised portions of said dielectric material length B and height C.

FIG. 11B shows an overhead view of a standard DEP stack. In accordance with the preferred embodiment of the present invention, the fingers or digits of the electrodes may extend laterally from a spine as shown. FIG. 11C shows a cross-section view of an etch block stack. In accordance with an alternative embodiment of the present invention, the semiconductor array may comprise an etch block stack as shown. In this embodiment, the semiconductor array containing the plurality of electrodes may comprise of four layers, a first metal layer, a third dielectric layer, a second dielectric layer, a first dielectric layer, and a substrate layer as shown. The first dielectric layer may be selected such that it resists the etch used for the third dielectric layer. The second dielectric layer may be etched to expose the first dielectric layer. FIG. 11D shows a cross-section of an edge capped stack. In accordance with an alternative embodiment of the present invention, the semiconductor array may be designed in an edge cap stack formation, wherein a dielectric cap of a second dielectric is disposed within a gap of a first dielectric.

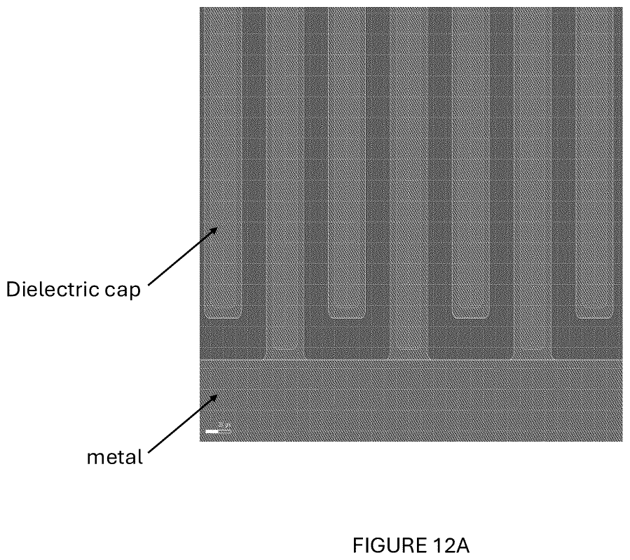

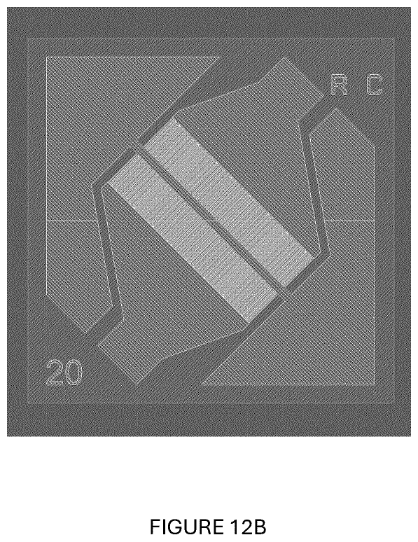

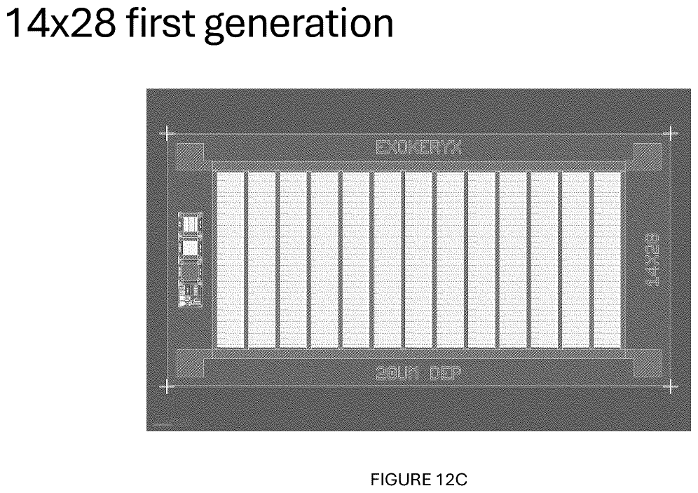

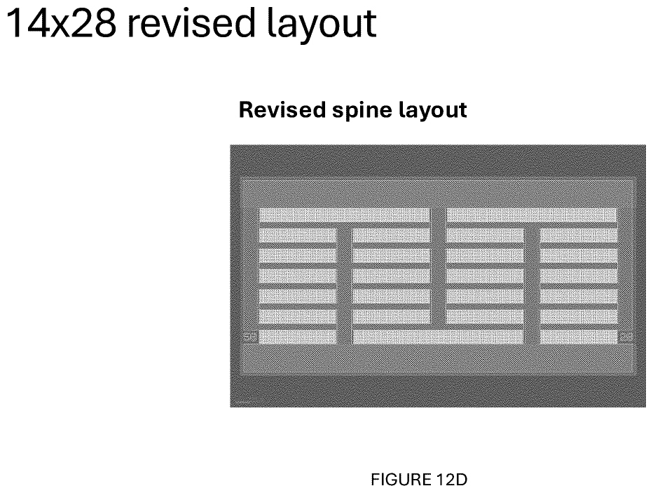

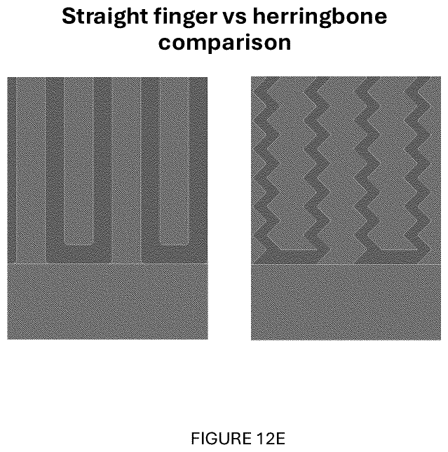

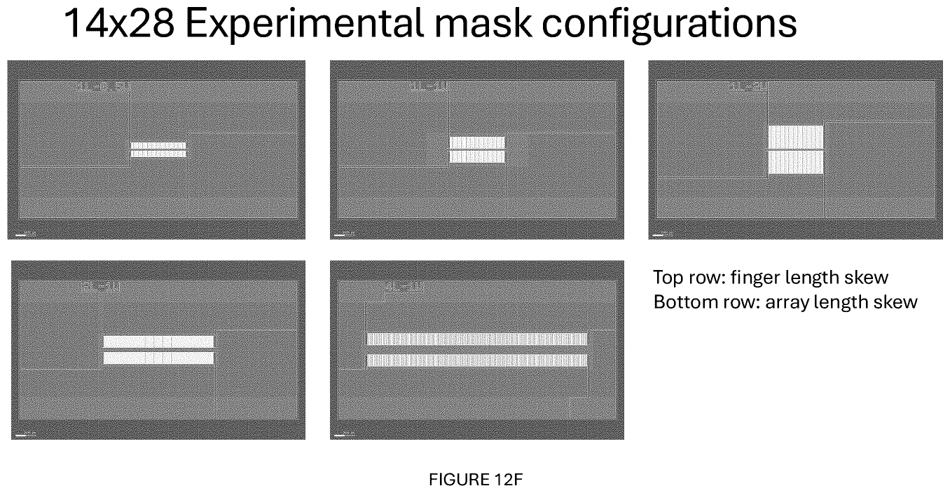

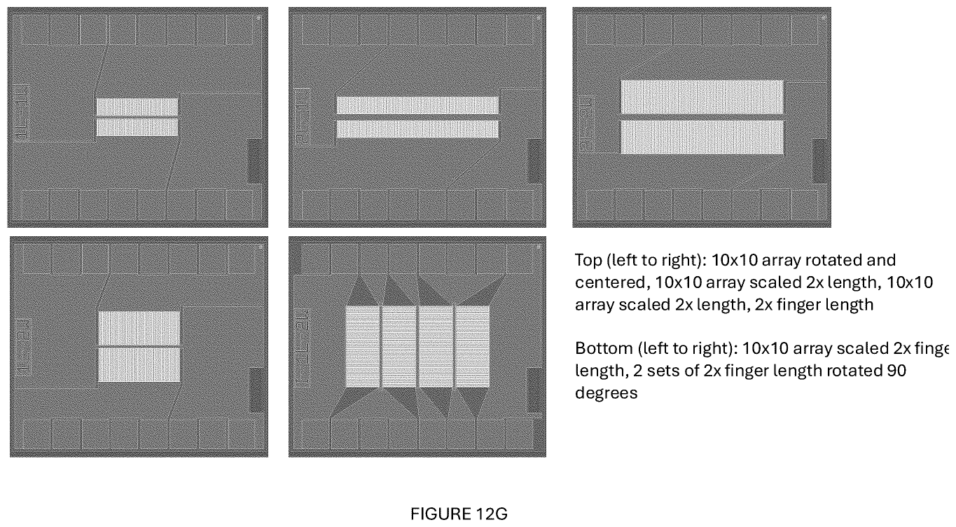

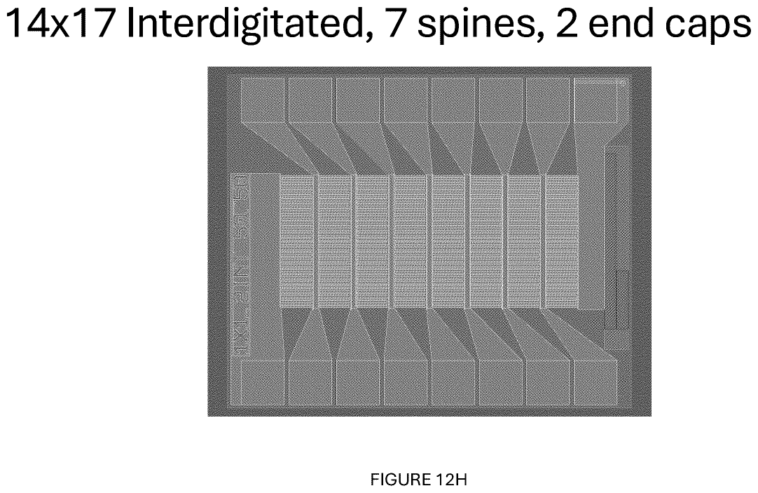

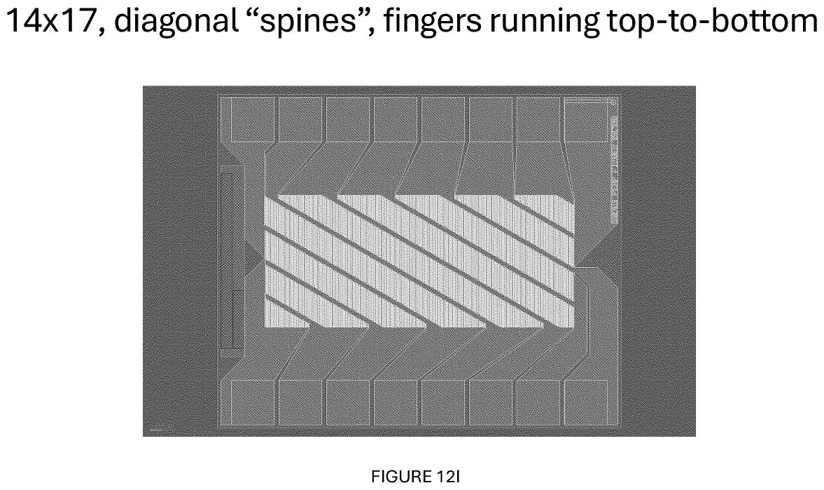

FIGS. 12A-H show variations of the semiconductor array. FIG. 12A shows an overhead view of an edge capped stack. In accordance with an alternative embodiment of the present invention, the interdigitated electrodes shown contain the dielectric cap as shown in FIG. 11D and as labelled herein. FIG. 12B shows a 10×10 diagonal flow cell with interdigitated fingers and electrical contacts in corners with various finger and gap spacings. The design presented herein can optionally contain dielectric caps around flow cell perimeter. FIG. 12C shows a 14×28 first generation cell. In accordance with an alternative embodiment of the present invention, the 14×28 first generation design shown may comprise top and/or bottom “bus bar” powered from opposite polarities. The design shown in FIG. 12C provides maximized active array area compared to total wafer area. FIGS. 12D-H show alternative 14×28 and 14×17 revised layout cells, including a revised spine layout (FIG. 12D), straight and herringbone finger designs (FIG. 12E), experimental mask configurations (FIGS. 12F-G), interdigitated, 7 spines, 2 end caps (FIG. 12H), and diagonal spines, fingers running top to bottom (FIG. 12I).

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that may be included in the disclosed technology. The disclosed technology is not restricted to the illustrated example architectures or configurations, but the desired features may be implemented using a variety of alternative architectures and configurations. Indeed, it will be apparent to one of skill in the art how alternative functional, logical or physical partitioning and configurations may be implemented to implement the desired features of the technology disclosed herein. Also, a multitude of different constituent module names other than those depicted herein may be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead may be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof, the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

What is claimed is:

1. A system for a fluidics cartridge comprising a semiconductor array for biomarker detection, the system comprising:
a cartridge body comprising:
a plurality of pipette tips;
a pierceable well cover;
a cartridge base comprising:
a plurality of reagent storage wells,
a tip storage area,
a sample inlet well,
an elution outlet well,
mating features for a flow cell,
a waste reservoir, and
a pogo pin access;
a flow cell comprising:
a flow cell base comprising:
a plurality of separate parts,
an inlet docking port,
an outlet docking port,
a pogo pin access,
mating features for a pressure-sensitive adhesive (PSA) spacer,
mating features for said flow cell base,
a dielectrophoresis (DEP) chip,
a PSA spacer,
a top sealing film, and
a bottom sealing film.

2. The system of claim 1, wherein said reagent storage wells comprise a plurality of separate components including a priming buffer, a wash buffer, and a rinse.

3. The system of claim 1, wherein said waste reservoir further comprises a sponge.

4. The system of claim 1, wherein said waste reservoir is positioned adjacent to said reagent storage wells.

5. The system of claim 1, wherein said reagent storage wells are positioned under the pierceable well cover.

6. The system of claim 1, wherein said flow cell further comprises an overmold configured to provide a seal.

7. The system of claim 1, wherein said flow cell further comprises heat stakes to provide temperature control to said flow cell.

8. A method for biomarker detection using a fluidics cartridge comprising a semiconductor array, the method comprising:
introducing a sample fluid into the cartridge;
priming the cartridge with a prime buffer to prepare it for fluid flow;
pumping and recirculating at least one fluid within a flow cell comprising the semiconductor array;
washing the cartridge with a wash buffer;
washing the cartridge with an elution buffer under capture conditions; and
eluting the cartridge with an elution buffer under release conditions.

9. The method of claim 8, wherein said at least one fluid is a biological sample.

10. The method of claim 8, wherein said at least one fluid is pumped across a dielectrophoresis chip in a back-and-forth movement.

11. The method of claim 10, wherein said pumping is accomplished via a pulling and release mechanism of a pipette.

12. The method of claim 8, wherein said at least one fluid is removed from said cartridge after said elution buffer is eluted.

13. The method of claim 8, wherein said priming, said washing, and said eluting are accomplished via a pipette, wherein said pipette picks up and dispenses said prime buffer, said fluid, said wash buffer, and said elution buffer, and wherein said pipette pulls each of said prime buffer, said fluid, said wash buffer, and said elution buffer through the cartridge.

14. The method of claim 8, wherein said cartridge comprises:
a plurality of reagent storage wells,
a tip storage area,
a sample inlet well,
an elution outlet well,
a handle,
mating features for a flow cell,
a waste reservoir, and
a pogo pin access,
a plurality of pipette tips; and
a pierceable well cover.

15. A system for a fluidics cartridge comprising a semiconductor array configured to receive and process a fluid for biomarker detection, the system comprising:
a cartridge body comprising:
a cartridge base comprising:
a plurality of reagent storage wells,
a tip storage area,
a sample inlet well,
an elution outlet well,
a handle,
mating features for a flow cell,
a waste reservoir, and
a pogo pin access,
a plurality of pipette tips; and
a pierceable well cover;
a flow cell comprising:
a plurality of heat stakes; and
a flow cell base comprising:
a plurality of separate parts,
an inlet docking port,
an outlet docking port,
a pogo pin access,
mating features for PSA spacer,
mating features for said flow cell base,
a dielectrophoresis chip comprising a semiconductor array configured from a plurality of electrodes, wherein each of said plurality of electrodes is composed of a layer of metal, layer of dielectric material, and a layer of substrate,
a PSA spacer,
a top sealing film, and
a bottom sealing film.

16. The system of claim 15, wherein said fluid is blood, ethylenediaminetetraacetic acid (EDTA) plasma, urine, cerebrospinal fluid, concentrated conditioned media, serum, saliva, or other bodily fluid.

17. The system of claim 15, wherein said semiconductor array is arranged such that a plurality of digits of electrodes extend laterally from a spine.

18. The system of claim 15, wherein said semiconductor array comprises an edge capped stack comprising at least one additional dielectric layer.

19. The system of claim 15, wherein said semiconductor array is designed in a herringbone finger design, an experimental mask configuration, interdigitated spines, end caps, and diagonal spines, or fingers arranged in a top-to-bottom configuration.

20. The system of claim 15, wherein said fluid is an input sample comprising an additive or dilutant.
